(19) United States
(12) Patent Application Publication
(10) Pub. No.: US 2008/0118940 A1
(43) Pub. Date: May 22, 2008

(54) PROTEASOMAL PEPTIDASE ACTIVITY AND THE USE THEREOF IN CLINICAL APPLICATIONS

(76) Inventor: Maher Albitar, Coto De Caza, CA (US)

Correspondence Address: FOLEY & LARDNER LLP, P.O. Box 80278, San Diego, CA, 92138-0278, US

(21) Appl. No.: 11/685,651

(22) Filed: Mar. 13, 2007

Related U.S. Application Data
(60) Provisional application No. 60/866,898, filed on Nov. 22, 2006.

(51) Int. Cl. C12Q 1/37

(52) U.S. Cl. 435/23; 435/24

(57) ABSTRACT

Provided herein are methods for the diagnosis, prognosis, or management of hematological disorders and other diseases using the proteasome activity levels determined in acellular body fluids or cell-containing samples. Also provided are methods of monitoring treatment with proteasome inhibitors through assaying proteasome activity in acellular body fluids. Further provided are methods of predicting response to therapy in certain populations of leukemia patients.

### CROSS-REFERENCE TO RELATED APPLICATIONS

This application claims the benefit of priority under 35 U.S.C. § 119(e) to U.S. Provisional Application Ser. No. 60/866,898 filed Nov. 22, 2006, which is incorporated by reference herein in its entirety including all figures and tables.

### FIELD OF THE INVENTION

The invention relates to medically useful assays, in particular, the determination of proteasome activity level and its use in the diagnosis, prognosis, and management of disease, including hematopoietic disorders.

### BACKGROUND OF THE INVENTION

The following discussion of the background of the invention is merely provided to aid the reader in understanding the invention and is not admitted to describe or constitute prior art to the present invention.

The ubiquitin-proteasome system is responsible for the degradation of approximately 80-90% of normal and abnormal intracellular proteins and therefore plays a central role in a large number of physiological processes. For example, the regulated proteolysis of cell cycle proteins, including cyclins, cyclin-dependent kinase inhibitors, and tumor suppressor proteins, is required for controlled cell cycle progression and proteolysis of these proteins occurs via the ubiquitin-proteasome pathway (Deshaies, Trends in Cell Biol. 5: 428-434 (1995) and Hoyt, Cell 91:149-151 (1997)). In another example, the activation of the transcription factor NF-κB, which itself plays a central role in the regulation of genes involved in the immune and inflammatory responses, is dependent upon the proteasome-mediated degradation of an inhibitory protein, Iκα B-α (Palombella et al., WO 95/25533). In yet another example, the ubiquitin-proteasome pathway plays an essential role in antigen presentation through the continual turnover of cellular proteins (Goldberg and Rock, WO 94/17816).

While serving a central role in normal cellular homeostasis, the ubiquitin-proteasome pathway also mediates the inappropriate or accelerated protein degradation occurring as a result or cause of pathological conditions including cancer, inflammatory diseases, and autoimmune diseases, characterized by deregulation of normal cellular processes. In addition, the cachexia or muscle wasting associated with conditions such as cancer, chronic infectious diseases, fever, muscle atrophy, nerve injury, renal failure, and hepatic failure results from an increase in proteolytic degradation by the ubiquitin-proteasome pathway (Goldberg, U.S. Pat. No. 5,340,736 (1994)). Furthermore, the cytoskeletal reorganization that occurs during maturation of protozoan parasites is proteasome-dependent (Gonzales et al., J. Exp. Med. 184:1909 (1996)).

Central to this system is the 26S proteasome, a multisubunit proteolytic complex, consisting of one 20S proteasome core and two flanking 19S complexes. The 20S proteasome consists of four rings: two outer α-rings and two inner β-rings surrounding a barrel-shaped cavity. The two inner β-rings form a central chamber that harbors the catalytic for the chymotryptic, tryptic, and caspase-like activities (von Mikecz, J Cell Sci, 119(10)1977-84, 2006).

Proteins targeted for degradation by the proteasome contain a recognition signal. This signal consists of a polyubiquitin chain that is selectively attached to the protein target by the sequential addition of ubiquitin monomers. The polyubiquitin signal is recognized by the 19S complex, which mediates the entry of the target protein into the proteolytic chamber.

Drugs that inhibit proteasome activity have been used to treat a number of diseases such as cancer, inflammatory disease, and autoimmune disease. Methods of monitoring proteasome inhibitor drug action, using an ex vivo assay of proteasome activity have been described (U.S. Pat. No. 6,613,541).

### SUMMARY OF THE INVENTION

The present invention is based on the discovery that proteasomal peptidase activity ray be detected in patient samples and that such activity can have clinical value such as diagnosis and prognosis of certain disease states, prediction of response to therapy, prognosis, and monitoring of proteasome inhibitor therapy.

In a first aspect, the invention provides methods of diagnosing a disease or disorder other than a pancreatic disease or disorder, cystic fibrosis, or renal failure in a subject. In one approach, the method includes,

determining the level of one or more proteasomal peptidase activities in a test sample from the subject, and

using the determined levels of the one or more proteasomal peptidase activities in conjunction with clinical factors other than proteasomal peptidase activity to diagnose the presence of the disease or disorder in the patient.

In another approach for the above aspect of the invention, the method of diagnosing a disease or disorder other than a pancreatic disease or disorder, cystic fibrosis, or renal failure in a subject includes,
determining in a test sample from the subject, a peptidase activity level for one or more substrates, wherein each of the substrates is cleaved by a peptidase activity selected from the group consisting of proteasomal chymotrypsin-like activity, (Ch-L), proteasomal trypsin-like activity (Tr-L), and proteasomal caspase-like activity (Cas-L), and
using the determined peptidase activity level for one or more substrates in conjunction with clinical factors other than proteasomal peptidase activity to diagnose the presence of the disease or disorder in the patient.

In preferred embodiments the proteasomal peptidase activities are selected from the group consisting of chymotrypsin-like activity (Ch-L), trypsin-like activity (Tr-L), and caspase-like activity (Cas-L).

In particular embodiments, an increase in the level of the one or more peptidase activities relative to the level of the corresponding peptidase activity in a comparable sample from one or more healthy individuals is used with clinical factors other than peptidase activity to diagnose the presence of the disease or disorder in the patient.

“Proteasomal peptidase activity” refers to any proteolytic enzymatic activity associated with a proteasome, such as the 26S or 20S proteasomes. The peptidase activities of proteasomes include, for example, chymotrypsin-like activity (Ch-L), trypsin-like activity (Tr-L), and caspase-like activity (Cas-L). In preferred embodiments, proteasomal peptidase activity is determined by measuring the rate of cleavage of a substrate per unit volume of body fluid assayed. Thus, the activity is preferably expressed as (moles of substrate)/time/(volume body fluid). For example, the activity may be expressed as pmol/sec/mL.

As used herein the term “proteasome” refers to certain large protein complexes within cells or body fluid that degrade proteins that have been tagged for elimination, particularly those tagged by ubiquitination. Proteasomes degrade denatured, misfolded, damaged, or improperly translated proteins. Proteasomal degradation of certain proteins, such as cyclins and transcription factors, serves to regulate the levels of such proteins. Exemplary proteasomes include the 26S proteasome, 20S proteasome, and the immunoproteasome.

The 26S proteasome consists of 3 subcomplexes. The 26S proteasome consists of a 20S proteasome at the core which is capped at each end by a 19S regulatory particle (RP or PA700). The 19S RP mediates the recognition of the ubiquitinated target proteins, the ATP-dependent unfolding and the opening of the channel in the 20S proteasome, allowing entry of the target protein into the proteolytic chamber.

The 20S proteasome, which forms the core protease (CP) of the 26S proteasome, is a barrel-shaped complex consisting of four stacked rings, each ring having 7 distinct subunits. The four rings are stacked one on top of the other and are responsible for the proteolytic activity of the proteasome. There are two identical outer a rings, having no known function, and two inner β rings, containing multiple catalytic sites. In eukaryotes, two of these sites on the β rings have chymotrypsin-like activity (Ch-L), two of these sites have trypsin-like activity (Tr-L), and two have caspase-like activity (Cas-L).

The immunoproteasome, which is characterized by an ability to generate major histocompatibility complex class I-binding peptides, consists of a 20S proteasome core capped on one end by 19S RP and on the other end by PA28, an activator of the 20S proteasome and an alternative RP. PA28 consists of two homologous subunits (termed α and β) and a separate but related protein termed PA28γ (also known as the Ki antigen).

The term “test sample” as used herein refers to a sample in which the proteasomal peptidase activity is determined. Test samples may be obtained from healthy individuals or patients or subjects undergoing diagnosis or treatment of a disease or disorder. Test samples may be cell-containing samples, or acellular liquid samples. Preferably test samples are body fluid samples, more preferably acellular body fluid samples. In some embodiments, test samples are cell-containing samples.

The term “body fluid” or “bodily fluid” as used herein refers to any fluid from the body of an animal. Examples of body fluids include but are not limited to plasma, serum, blood, lymphatic fluid, cerebrospinal fluid, synovial fluid, urine, saliva, mucous, phlegm and sputum. Plasma and serum are preferred body fluids of the present invention. A body fluid sample of the present invention may be collected by any suitable method. The body fluid sample may be used immediately or may be stored for later use. Any suitable storage method known in the art may be used to store the body fluid sample; for example the sample may be frozen at about −20° C. to about −70° C. Preferred body fluids are acellular fluids. “Acellular” fluids include body fluid samples in which cells are absent or are present in such low amounts that the peptidase activity level determined reflects its level in the liquid portion of the sample, rather than in the cellular portion. Preferably an acellular body fluid contains no intact cells. Examples of acellular fluids include plasma or serum, or body fluids from which cells have been removed. In preferred embodiments, plasma is the acellular fluid.

In certain embodiments, the proteasome activity is determined in the cells of, for example, a “cell-containing sample”. In these embodiments, a cell-containing sample affected by a disease or disorder may be used. Preferred cell-containing samples include samples of tumor cells, blood cells, peripheral blood lymphocytes (PBLs), and white blood cells (WBCs).

The term “comparable sample” in the context of comparing two or more samples, means that the same type of sample (e.g., plasma) is used in the comparison. For example, a activity level in a sample of plasma can be compared to an activity level in another plasma sample. In some embodiments, comparable samples may be obtained from the same individual at different times. In other embodiments, comparable samples may be obtained from different individuals (e.g., a patient and a healthy individual). In general, comparable samples are normalized by a common factor. For example, acellular body fluid samples are normalized by volume body fluid and cell-containing samples are normalized by protein content or cell count.

The term “diagnose” as used herein refers to the act or process of identifying or determining a disease or condition in a mammal or the cause of a disease or condition by the evaluation of the signs and symptoms of the disease or disorder. Usually, a diagnosis of a disease or disorder is based on the evaluation of one or more clinical factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease.

“Clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes (e.g., liver enzymes), examination of blood cells or bone marrow cells, cytogenetics, and immunophenotyping of blood cells. Proteasome activity is a clinical factor.

The term “using the determined levels of one or more proteasomal peptidase activities” as used herein in reference to the diagnosis of disease, refers to comparing the peptidase activity level in a sample from a patient to the presence or amount of the level determined in a comparable sample from persons known to suffer from, or known to be at risk of, a given condition; or in persons known to be free of a given condition. Likewise, the term as used herein in reference to determining a patient's prognosis, refers to comparing the peptidase activity level in a sample from a patient diagnosed with a particular disorder, to the presence or amount of the level determined in a comparable sample from patients diagnosed with the same disorder for whom the outcome of the disorder is known. In certain embodiments of the invention, a threshold level of proteasome activity can be established for a given diagnosis or prognosis, and the level of proteasome activity in a patient sample can simply be compared to the threshold level.

In some embodiments of the above aspect of the invention, the disease or disorder may be a cancer, a proliferative hematological disorder, an inflammatory disease, an autoimmune disease, or a degenerative disease.

In certain embodiments of the above aspects of the invention, the level of one or more proteasomal peptidase activities in a test sample from a patient is used in the diagnosis of cancer. Cancer is a class of diseases characterized by uncontrolled cell division and the ability of these cells to invade other tissues, either by direct growth into adjacent tissue (invasion) or by migration of cells to distant sites (metastasis). Cancer cells may spread throughout the body (i.e., metastasize) by way of the bloodstream or lymphatic system to form tumors in other tissues or organs. Such cancers include, but are not limited to leukemia, lymphoma, breast cancer, lung cancer, esophageal cancer, stomach cancer, colorectal cancer, thyroid cancer, melanoma, bone cancer, prostate cancer, testicular cancer, ovarian cancer, cervical cancer, endometrial cancer, kidney cancer, bladder cancer, and cancer of the central nervous system. Preferably the cancer is not pancreatic cancer.

In other embodiments, the level of one or more proteasomal peptidase activities in a test sample from a patient is used in the diagnosis of a proliferative hematological disorder. The term “proliferative hematological disorder” as used herein means a disorder of a bone marrow or lymph node derived cell type such as a white blood cell. A proliferative hematologic disorder is generally manifest by abnormal cell division resulting in an abnormal level of a particular hematological cell population. The abnormal cell division underlying a proliferative hematological disorder is preferably inherent in the cells and not a normal physiological response to infection or inflammation. A leukemia is a type of proliferative hematological disorder. Exemplary proliferative hematological disorders include, but are not limited to, acute myeloid leukemia, acute lymphoblastic leukemia, chronic lymphocytic leukemia, myelodysplastic syndrome, chronic myeloid leukemia, hairy cell leukemia, leukemic manifestations of lymphomas, and multiple myeloma. Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma). Infection or acute inflammation resulting in a transient spike in circulating white blood cells is preferably not included within the meaning of a proliferative hematologic disorder.

In certain embodiments of the above aspect of the invention, an increase in the level of the one or more proteosomal peptidase activities relative to the level of the corresponding proteosomal peptidase activity in a comparable sample from one or more healthy individuals is a factor favoring diagnosis of a proliferative hematological disorder while the same or a decrease in the level of the one or more proteosomal peptidase activities relative to the level of the corresponding proteosomal peptidase activity in a comparable sample from one or more healthy individuals is a factor against diagnosis of a proliferative hematological disorder. In particular embodiments, an increase in the levels of activity of one or more of Ch-L, Tr-L, and Cas-L activities are useful for the diagnosis of chronic lymphocytic leukemia (CLL), acute myeloid leukemia (AML) myelodysplastic syndrome (MDS), or acute lymphoblastic leukemia.

In other embodiments of the above aspects of the invention, the level of one or more proteasomal peptidase activities in a test sample from a patient are used in the diagnosis of an inflammatory disease. “Inflammatory disease” as used herein generally refers to chronic conditions in which a tissue is inflamed. Inflammation is characterized by pain, swelling, redness, and heat. Examples of inflammatory diseases include rheumatoid arthritis, osteoarthritis, inflammatory lung disease, inflammatory bowel disease, atherosclerosis, and psoriasis.

In still other embodiments, the level of one or more proteasomal peptidase activities in a test sample from a patient is used in the diagnosis of an autoimmune disease. Autoimmune diseases are those conditions in which the body produces an immune response against self antigens. Such anti-self responses may include the formation of antibodies. Examples of autoimmune diseases include, but are not limited to, diabetes mellitus type 1, systemic lupus erythematosus (SLE), Sjögren's syndrome, Hashimoto's thyroiditis, rheumatoid arthritis (RA), scleroderma, polymyositis, Addison's disease, autoimmune hemolytic anaemia, and multiple sclerosis.

In further embodiments, the level of one or more proteasomal peptidase activities in a test sample from a patient is used in the diagnosis of a degenerative disease. Degenerative diseases are those conditions in which the function or structure of the affected tissues or organs progressively deteriorate over time. Examples of degenerative diseases include, but are not limited to, Alzheimer's disease, amyotrophic lateral sclerosis (ALS) (e.g., Lou Gehrig's disease), atherosclerosis, cancer, diabetes, heart disease, inflammatory bowel disease (IBD), Parkinson's disease, prostatitis, osteoarthritis, and osteoporosis.

In another aspect of the invention, there are provided methods of predicting the response to therapy of a leukemia patient having intermediate-risk cytogenetic abnormalities. In one approach, the method includes

determining the level of activity for proteasomal caspase-like activity (Cas-L) in a test sample from the patient, and

using the level of activity to predict the response of the patient to therapy.

In another approach of the above aspect of the invention, the method of predicting the response to therapy of a leukemia patient having intermediate-risk cytogenetic abnormalities includes

determining in a test sample from the patient a peptidase activity level for one or more substrates, wherein each of the substrates is cleaved by proteasomal caspase-like activity (Cas-L), and

using the level of activity to predict the response of the patient to therapy.

In certain embodiments, the leukemia patient has AML, CLL, or ALL. In preferred embodiments the leukemia patient has AML.

In preferred embodiments, the level of Cas-L activity is compared to a cutoff value determined from the level of Cas-L activity present in a comparable sample from one or more healthy individuals, and wherein an increase or decrease in the patient value relative to the cutoff value is used to predict the response of the patient to therapy.

In some embodiments, the cutoff value is 1.0, 1.1, 1.2, 1.3, 1.4, 1.5, 2.0, 2.5, 3.0, 3.5, 4.0, 5.0, 6.0, 7.0, 8.0, 9.0, or 10.0. In some embodiments the cutoff value is 1.0, 1.1, 1.2, 1.3, 1.4, or 1.5. In other embodiments the cutoff value is 3.0, 3.5, 4.0, 5.0, 6.0, or 7.0. In preferred embodiments of the above aspect of the invention the cutoff value is 3 pmol AMC/sec/mL plasma and Cas-L activity greater than the cutoff is associated with a poor response to chemotherapy.

The term “patient,” as used herein refers to an individual who is under the care of a medical practitioner, an individual who is ill, or an individual who is affected with a disease or disorder. Preferably, patients of the present invention are humans; however, animals that are ill, under veterinarian care or affected by a disease or disorder may also be patients as used herein. The term “MDS patient” as used herein refers to a patient diagnosed with MDS. One of ordinary skill in the art is capable of diagnosing MDS using suitable diagnostic criteria. Likewise, the term “AML patient,” “CLL patient,” “ALL patient,” and “CML patient” refers to a patient diagnosed with AML, CLL, ALL, or CML, respectively.

Cytogenetics refers to the analysis of the physical appearance of chromosomes (e.g., the number and shape of the chromosomes). The identification of particular chromosome alterations or abnormalities can be helpful in diagnosing, for example, specific types of leukemia and lymphoma. Furthermore, particular types of chromosomal alterations have been associated with clinical behavior or response to therapy and therefore can be used in determining treatment approaches. For example, patients with AML are assigned to one of several risk categories (i.e., good, intermediate, bad, and very bad) based on the appearance of metaphase chromosomes according to the International System for Cytogenetic Nomenclature (ISCN). Thus, in AML, patients in the good-risk category or having good cytogenetics exhibit t(8; 21) or inv16; intermediate-risk or intermediate cytogenetics exhibit a normal karyotype (NN) or —Y only; the bad-risk category or bad cytogenetics include all other abnormalities, without good and very bad cytogenetic features; and very bad risk -5, 5q-, -7, 7q-, complex abnormalities (i.e., clones with a number of unrelated abnormalities), abnormal (abn) 3q, t(9; 22), t(6; 9), or abn 11q23 and absence of good cytogenetic features.

In still another aspect of the invention there are provided methods of monitoring proteasome inhibitor drug therapy in a patient. In one approach the method includes,

obtaining a sample of an acellular body fluid from a patient previously administered a proteasome inhibitor;

determining the level of one or more proteasomal peptidase activities in a sample of an acellular body fluid from the patient, the peptidase activities selected from the group consisting of chymotrypsin-like activity (Ch-L), trypsin-like activity (Tr-L), and caspase-like activity (Cas-L), and
comparing the level of activity in the sample to a control level of activity for each peptidase determined in a comparable sample of acellular body fluid obtained from the patient prior to the administration of the proteasome inhibitor or obtained from a one or more healthy individuals.

In another approach of the above aspect of the invention, the method of monitoring proteasome inhibitor drug therapy in a patient includes,

obtaining a sample of an acellular body fluid from a patient previously administered a proteasome inhibitor;

determining in a the acellular body fluid sample a peptidase activity level for one or more substrates, wherein each of the substrates is cleaved by a peptidase activity selected from the group consisting of proteasomal chymotrypsin-like activity, (Ch-L), proteasomal trypsin-like activity (Tr-L), and proteasomal caspase-like activity (Cas-L), and
comparing the level of activity in the sample to a control level of activity for each peptidase determined in a comparable sample of acellular body fluid obtained from the patient prior to the administration of the proteasome inhibitor or obtained from a one or more healthy individuals.

In preferred embodiments, the control level of activity for each peptidase is determined in a comparable sample of acellular body fluid obtained from the patient prior to the initiation of proteasome inhibitor therapy.

In other embodiments, levels of proteasomal peptidase activity may be determined for acellular body fluid samples obtained from the patient at one or more time points following administration of the proteasome inhibitor.

“Proteasome inhibitor” as used herein refers to any substance which directly or indirectly inhibits the 20S or 26S proteasome or the activity thereof. Non-limiting examples of proteasome inhibitors include peptide aldehydes (see, e.g., Stein et al. WO 95/24914 published Sep. 21, 1995; Siman et al. WO 91/13904 published Sep. 19, 1991; Iqbal et al. J. Med. Chem. 38:2276-2277 (1995)), vinyl sulfones (see, e.g., Bogyo et al., Proc. Natl. Acad. Sci. 94:6629 (1997)), alpha ‘beta’-epoxyketones (see, e.g., Spaltenstein et al. Tetrahedron Lett. 37:1343 (1996)); peptide boronic acids (see, e.g., Adams et al. WO 96/13266 published May 9, 1996; Siman et al. WO 91/13904 published Sep. 19, 1991), and lactacystin and lactacystin analogs (see, e.g., Fenteany et al. Proc. Natl. Acad. Sci. USA 94:3358 (1994); Fenteany et al. WO 96/32105 published Oct. 19, 1996).

In yet another aspect of invention there are provided methods of determining a prognosis of a patient having a disease or disorder. In one approach the method includes,
determining the level of one or more proteasomal peptidase activities in a test sample from the subject, the peptidase activities selected from the group consisting of chymotrypsin-like activity (Ch-L), trypsin-like activity (Tr-L), and caspase-like activity (Cas-L), and
using the levels of the one or more proteasomal peptidase activities determined for the patient in conjunction with clinical factors other than proteasomal peptidase activity to determine a prognosis for the patient.

In an other approach of the above aspect of invention, the method of determining a prognosis of a patient having a disease or disorder includes,

determining in a test sample from the patient a peptidase activity level for one or more substrates, wherein each of the substrates is cleaved by a peptidase activity selected from the group consisting of proteasomal chymotrypsin-like activity, (Ch-L), proteasomal trypsin-like activity (Tr-L), and proteasomal caspase-like activity (Cas-L), and
using the levels of the one or more proteasomal peptidase activities determined for the patient in conjunction with clinical factors other than proteasomal peptidase activity to determine a prognosis for the patient.

In some embodiments of the above aspect of the invention, the disease or disorder is preferably a disease or disorder other than a pancreatic disease or disorder, cystic fibrosis, or renal failure.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of years, on average, that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense a “favorable prognosis” an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a lower propensity for metastasis, a longer than expected life expectancy, differentiation of a benign process from a cancerous process, and the like. For example, a positive prognosis is one where a patient has a 50% probability of being cured of a particular cancer after treatment, while the average patient with the same cancer has only a 25% probability of being cured. A positive prognosis in cancer may be indicated by, for example, chemical destruction of a tumor vasculature.

A prognosis is often determined by examining one or more clinical factors and/or symptoms that correlate to patient outcomes. As described herein, the activity level of a proteasomal peptidase is a clinical factor useful in determining prognosis. The skilled artisan will understand that associating a clinical factor with a predisposition to an adverse outcome may involve statistical analysis.

Additionally, a change in a clinical factor from a baseline level may impact a patient's prognosis, and the degree of change in level of the clinical factor may be related to the severity of adverse events. Statistical significance is often determined by comparing two or more populations, and determining a confidence interval and/or a p value. As used herein a “confidence interval” or “CI” refers to a measure of the precision of an estimated or calculated value. The interval represents the range of values, consistent with the data, that is believed to encompass the “true” value with high probability (usually 95%). The confidence interval is expressed in the same units as the estimate or calculated value. Wider intervals indicate lower precision; narrow intervals indicate greater precision. Preferred confidence intervals of the invention are 90%, 95%, 97.5%, 98%, 99%, 99.5%, 99.9% and 99.99%. A “p-value” as used herein refers to a measure of probability that a difference between groups happened by chance. For example, a difference between two groups having a p-value of 0.01 (or p=0.01) means that there is a 1 in 100 chance the result occurred by chance. Preferred p values are 0.1, 0.05, 0.025, 0.02, 0.01, 0.005, 0.001, and 0.0001. Confidence intervals and p-values can be determined by methods well-known in the art. See, e.g., Dowdy and Wearden, Statistics for Research, John Wiley & Sons, New York, 1983. Exemplary statistical tests for associating a prognostic indicator with a predisposition to an adverse outcome are described hereinafter.

Multiple determinations of proteasomal peptidase activity levels can be made, and a temporal change in activity can be used to determine a diagnosis or prognosis. For example, comparative measurements are made of the proteasomal peptidase activity of an acellular body fluid in a patient at multiple time points, and a comparison of a proteasomal peptidase activity value at two or more time points may be indicative of a particular diagnosis or prognosis.

In preferred embodiments of the above aspect of the invention, the determined peptidase activity levels can be compared to a reference value. In some embodiments, the reference value for each peptidase activity can be the level of activity for each peptidase in a comparable sample from one or more healthy individuals. In certain embodiments, elevated levels of one or more proteasomal peptidase activities relative to the corresponding reference value correlate with a negative prognosis. In a particular embodiment, the reference value is a cutoff value that has been statistically calculated based on peptidase activities determined from a particular population of individuals (e.g., a population of AML patients) or based on a statistical model to determine a cutoff value for predicting a specific clinical behavior. In this embodiment, a determined level of one or more proteasomal peptidase activities greater than a cutoff value is related to an unfavorable prognosis for the patient. In some embodiments the cutoff value is 1.0, 1.1, 1.2, 1.3, 1.4, 1.5, 1.6, 1.7, 1.8, 1.9, 1.96, 2.0, 2.1, 2.2, 2.3, 2.4, 2.5, 3.0, 4.0, 5.0, 6.0, 7.0, 8.0, 9.0, or 10.0. In preferred embodiments, the cutoff value is 1.96. In other embodiments, a determined level of one or more proteasomal peptidase activity in the patient sample that is the same as or substantially the same as the level in the reference sample (i.e., a comparable acellular body fluid sample from one or more healthy individuals) reflects a positive prognosis for the patient.

The phrase “substantially the same as” in reference to a comparison of one value to another value for the purposes of clinical management of a disease or disorder means that the values are statistically not different. Differences between the values can vary, preferably one value is within 20% of the other value, more preferably within 10%, and most preferably within 5% of the other value.

In particular embodiments, prognosis is the survival rate, preferably the 5 year survival rate. In other embodiments the prognosis is complete remission duration (CRD).

In some embodiments, the hematological disorder is CLL, and an elevation in chymotrypsin-like (Ch-L) or caspase-like (Cas-L) activity as compared to the reference value is associated with a decrease in survival, wherein the reference value is derived from a sample from one or more healthy volunteers. In other embodiments, the reference values for Ch-L and Cas-L activities are the median value of the corresponding activity in a population of patients having CLL. In these embodiments, an activity higher than the median value is associated with a decrease in survival.

In other embodiments, the hematological disorder is AML and an elevation in chymotrypsin-like (Ch-L) and caspase-like (Cas-L) activity as compared to a reference value derived from a sample from one or more healthy volunteers is associated with a decrease in survival. In other embodiments, an elevation in caspase-like (Cas-L) activity as compared to a median value of Cas-L activity in a population of patients having AML is associated with a decrease in survival.

In still other embodiments, a patient having AML and exhibiting intermediate-risk cytogenetic abnormalities, a caspase-like (Cas-L) activity higher than a reference value of 3 pmol AMC/sec/mL plasma, wherein a Cas-L higher than the reference value is associated with a decrease in survival.

In yet other embodiments, the hematological disorder is MDS and caspase-like (Cas-L) activity as compared to a reference value derived from the median value of Cas-L activity in a population of patients having MDS is associated with a decrease in survival.

In further embodiments, the hematological disorder is ALL and an elevation in caspase-like (Cas-L) activity as compared to a cutoff value derived from the median value of Cas-L activity in a population of patients having ALL is associated with longer survival. In some embodiments, the cutoff value is 1.5, 2.0, 2.5, 2.7, 3.0, 3.5, 4.0, 5.0, 6.0, 7.0, 8.0, 9.0, or 10.0 pmol AMC/sec/mL plasma. In preferred embodiments, the patient's Cas-L activity is compared to a cutoff value of 2.7 pmol AMC/sec/mL plasma and a Cas-L activity higher than the cutoff value is associated with longer survival.

The term “about” as used herein in reference to quantitative measurements or values, refers to the indicated value plus or minus 10%.

### BRIEF DESCRIPTION OF THE FIGURES

FIG. 1 shows the chymotrypsin-like (“Chymo”), trypsin-like (“Tryp”), and caspase-like (“Casp”) activity of purified proteasomes in the presence (grey bars) and absence (stippled bars) of proteasome inhibitor YU-101 (“IHB”).

FIG. 2 shows the chymotrypsin-like (“Chymo”), trypsin-like (“Tryp”), and caspase-like (“Casp”) activity determined in a sample of plasma in the presence (grey bars) and absence (stippled bars) of proteasome inhibitor YU-101 (“IHB”).

FIG. 3 shows the chymotrypsin-like (“Chymo”), trypsin-like (“Tryp”), and caspase-like (“Casp”) activity determined in a white blood cell lysate in the presence (grey bars) and absence (stippled bars) of proteasome inhibitor YU-101 (“IHB”).

### DETAILED DESCRIPTION OF THE INVENTION

Sample Preparation

Provided herein are methods of using the information obtained through analysis of the level of activity of one or more peptidase activities of proteasomes in test samples of acellular body fluid or cell-containing samples. Test samples may be obtained from an individual or patient. Methods of obtaining test samples are well-known to those of skill in the art and include, but are not limited to, aspirations or drawing of blood or other fluids. Samples may include, but are not limited to, whole blood, serum, plasma, saliva, cerebrospinal fluid (CSF), pericardial fluid, pleural fluid, urine, and eye fluid.

In embodiments in which the proteasome activity will be determined using an acellular body fluid, the test sample may be obtained from a person may be a cell-containing liquid or an acellular body fluid (e.g., plasma or serum). In some embodiments in which the test sample contains cells, the cells may be removed from the liquid portion of the sample by methods known in the art (e.g., centrifugation) to yield acellular body fluid for the proteasome activity measurement. In preferred embodiments serum or plasma are used as the acellular body fluid sample; more preferably plasma. Plasma and serum can be prepared from whole blood using suitable methods well-known in the art. In these embodiments, data may be normalized by volume of acellular body fluid.

In other embodiments, the proteasomal peptidase activity is determined using a cell-containing sample. In these embodiments the cell-containing sample includes, but is not limited to, blood, urine, organ, and tissue samples. Preferably, the cell-containing sample is a blood sample, more preferably a blood cell lysate. Cell lysis may be accomplished by standard procedures. In certain preferred embodiments, the cell-containing sample is a whole blood cell lysate. Kahn et al. (Biochem. Biophys. Res. Commun., 214:957-962 (1995)) and Tsubuki et al. (FEBS Lett., 344:229-233 (1994)) disclose that red blood cells contain endogenous proteinaceous inhibitors of the proteasome. However, endogenous proteasomal peptidase inhibitors can be inactivated in the presence of SDS at a concentration of about 0.05%, allowing red blood cell lysates and whole blood cell lysates to be assayed reliably. At this concentration of SDS, most if not all proteasomal peptidase activity is due to the 20S proteasome. Although purified 20S proteasome exhibits poor stability at 0.05% SDS, 20S proteasomal peptidase activity in cell lysates is stable under these conditions (Vaddi et al. U.S. Pat. No. 6,613,541).

In certain other embodiments, the cell-containing sample is a white blood cell lysate. Methods for obtaining white blood cells from blood are known in the art (Rickwood et al., Anal. Biochem. 123:23-31 (1982); Fotino et al., Ann. Clin. Lab. Sci. 1:131 (1971)). Commercial products useful for cell separation include without limitation Ficoll-Paque (Pharmacia Biotech) and NycoPrep (Nycomed). In some situations, white blood cell lysates provide better reproducibility of data than do whole blood cell lysates and, therefore, may be preferred in those situations.

Variability in sample preparation of cell-containing samples can be corrected by normalizing the data by, for example, protein content or cell number. In certain embodiments, proteasomal peptidase activity in the sample may be normalized relative to the protein content in the sample (specific activity method). Total protein content in the sample can be determined using standard procedures, including, without limitation, Bradford assay and the Lowry method. In other embodiments, proteasomal peptidase activity in the sample may be normalized relative to cell number.

Measuring Proteasome Activity

Proteasome activity in the test sample can be measured by any assay method suitable for determining 20S or 26S proteasome activity. (See, e.g., Vaddi et al., U.S. Pat. No. 6,613,541; McCormack et al., Biochemistry 37:7792-7800 (1998)); Driscoll and Goldberg, J. Biol. Chem. 265:4789 (1990); Orlowski et al., Biochemistry 32:1563 (1993)). Preferably, a substrate having a detectable label is provided to the reaction mixture and proteolytic cleavage of the substrate is monitored by following disappearance of the substrate or appearance of a cleavage product. Detection of the label may be achieved, for example, by fluorometric, colorimetric, or radiometric assay.

Substrates for use in determining proteasomal peptidase activity may be chosen based on the selectivity of each peptidase activity. For example, the chymotrypsin-like peptidase preferentially cleaves peptides on the carboxyl side of tyrosine, tryptophan, phenylalanine, leucine, and methionine residues. The trypsin-like peptidase preferentially cleaves peptides on the carboxyl side of arginine and lysine residues. The caspase-like peptidase (or peptidylglutamyl-peptide hydrolase) preferentially cleaves peptides at glutamic acid and aspartic acid residues. Based on these selectivities, the skilled artisan can choose a specific substrates for each peptidase.

Preferred substrates for determining 26S proteasome activity include, without limitation, lysozyme, alpha-lactalbumin, beta-lactoglobulin, insulin b-chain, and ornithine decarboxylase. When 26S proteasome activity is to be measured, the substrate is preferably ubiquitinated or the reaction mixture preferably further contains ubiquitin and ubiquitination enzymes.

More preferably, the substrate is a peptide less than 10 amino acids in length. In one preferred embodiment, the peptide substrate contains a cleavable fluorescent label and release of the label is monitored by fluorometric assay. Non-limiting examples of substrates to measure trypsin-like activity include N—(N-benzoylvalylglycylarginyl)-7-amino-4-methylcoumarin (Bz-Val-Gly-Arg-AMC), N—(N-carbobenzyloxycarbonylleucylleucylarginyl)-7-amino-4-methylcoumarin (Z-Leu-Leu-Arg-AMC), Ac-Arg-Leu-Arg-AMC, and Boc-Leu-Arg-Arg-AMC. Non-limiting examples of substrates to measure caspase-like activity include N—(N-carbobenzyloxycarbonylleucylleucylglutamyl)-2-naphthylamine(Z-Leu-Leu-Glu-2NA), N—(N-carbobenzyloxycarbonylleucylleucylglutamyl)-7-amino-4-methylcoumarin (Z-Leu-Leu-Glu-AMC), and acetyl-L-glycyl-L-prolyl-L-leucyl-L-aspartyl-methylcoumarin (Ac-Gly-Pro-Leu-Asp-AMC). Non-limiting examples of substrates to measure chyrmotrypsin-like activity include N—(N-succinylleucylleucylvalyltyrosyl)-7-amino-4-methylcoumarin (Suc-Leu-Leu-Val-Tyr-AMC), Z-Gly-Gly-Leu-2NA, Z-Gly-Gly-Leu-AMC, and Suc-Arg-Pro-Phe-His-Leu-Leu-Val-Tyr-AMC.

Preferred substrates for measuring the chymotrypsin-like, caspase-like, and trypsin-like activities of the proteasome are Suc-Leu-Leu-Val-Tyr-AMC, Z-Leu-Leu-Glu-AMC, and Bz-Val-Gly-Arg-AMC, respectively, and the release of the cleavage product, AMC, can be monitored at 440 nm (λₑₓ=380 nm). Cleavage due to a particular peptidase may be determined by, for example, using a substrate specific for that peptidase and assaying that activity independent of other peptidases.

In certain preferred embodiments, the reaction mixture further contains a 20S proteasome activator. Preferred activators include those taught in Coux et al. (Ann. Rev. Biochem. 65: 801-847 (1995)), preferably PA28 or sodium dodecyl sulfate (SDS). However, SDS is not compatible with Bz-Val-Gly-Arg-AMC, therefore when Bz-Val-Gly-Arg-AMC is chosen as the substrate, PA28 is used instead of SDS to activate the proteasome.

Diagnosis of a Disease State

In preferred embodiments, the level of activity of one or more proteasomal peptidases in a test sample is used in conjunction with clinical factors other than proteasomal peptidase activity to diagnose a disease. In these embodiments, the level of proteasome activity measured in the test sample is compared to a reference value to determine if the levels of activity are elevated or reduced relative to the reference value. Preferably, the reference value is the proteasomal peptidase activity measured in a comparable sample from one or more healthy individuals. An increase or decrease in proteasome activity may be used in conjunction with clinical factors other than proteasomal peptidase activity to diagnose a disease.

The term “elevated levels” or “higher levels” as used herein refers to levels of a proteasome peptidase activity, that are higher than what would normally be observed in a comparable sample from control or normal subjects (i.e., a reference value). In some embodiments of the invention “control levels” (i.e. normal levels) refer to a range of proteasome activity levels that would be normally be expected to be observed in a mammal that does not have a hematological disorder and “elevated levels” refer to proteasome activity levels that are above the range of control levels. The ranges accepted as “elevated levels” or “control levels” are dependant on a number of factors. For example, one laboratory may routinely determine absolute levels of an activity of an enzyme in a sample that are different than the absolute levels obtained for the same sample by another laboratory. Also, different assay methods may achieve different value ranges. Value ranges may also differ in various sample types, for example different body fluids or by different treatments of the sample. One of ordinary skill in the art is capable of considering the relevant factors and establishing appropriate reference ranges for “control values” and “elevated values” of the present invention. For example, a series of samples from control subjects and subjects diagnosed with hematological disorders can be used to establish ranges that are “normal” or “control” levels and ranges that are “elevated” or “higher” than the control range.

Similarly, “reduced levels” or “lower levels” as used herein refer to levels of a proteasome peptidase activity that are lower than what would normally be observed in a comparable sample from control or normal subjects (i.e., a reference value). In some embodiments of the invention “control levels” (i.e. normal levels) refer to a range of proteasome activity levels that would be normally be expected to be observed in a mammal that does not have a hematological disorder and “reduced levels” refer to proteasome activity levels that are below the range of such control levels.

For example, elevated levels of Ch-L, Tr-L, and Cas-L activity as compared to the corresponding reference values from healthy individuals are associated with the presence of AML, ALL, CLL, and MDS. While an elevation in any of these proteasome activities alone does not provide a conclusive diagnosis, the information is useful in conjunction clinical factors other than proteasomal peptidase activity commonly used in the diagnosis of, for example, leukemia; the proteasome activity then provides a further factor in confirming a diagnosis.

Clinical factors of particular relevance in the diagnosis of hematological disorders include, but are not limited to, the patient's medical history, a physical examination of the patient, complete blood count, examination of bone marrow cells, cytogenetics, and immunophenotyping of blood cells.

Monitoring of Proteasome Inhibitor Activity

In one aspect of the invention, the level of proteasome activity in acellular body fluid of a patient is used to monitor treatment with proteasome inhibitors. In preferred embodiments, the level of a proteasomal peptidase activity in a test sample of acellular body fluid obtained from a patient treated with a proteasome inhibitor, can be compared to the activity level from a reference sample obtained from that patient prior to initiation of proteasome inhibitor treatment. Clinical monitoring of proteasome inhibitor drug action presently preferably entails this embodiment of the invention, with each patient serving as his or her own baseline control.

A decrease in proteasome activity in the patient test sample as compared to the patient's reference sample is indicative of an in vivo effect of the proteasome inhibitor at the time the test sample was obtained. In some embodiments, test samples are obtained at multiple timepoints following administration of the proteasome inhibitor. In these embodiments, measurement of proteasome activity in the test samples provides an indication of the extent and duration of in vivo effect of the proteasome inhibitor.

In certain embodiments, the amount of change in the proteasome activity can be used to calculate a dosage of a proteasome inhibitor and/or a dosing frequency. For example, the dose amount and dose frequency of the proteasome inhibitor can be selected so as to avoid excessive proteasome inhibition. Excessive proteasome inhibition can result in a toxic effect, the toxic effect including, but not being limited to, vomiting, diarrhea, hypovolemia, hypotension, and lethality.

In other embodiments, the dose amount and dose frequency of the proteasome inhibitor are selected so that therapeutically useful proteasome inhibition is achieved. Preferably, therapeutically useful proteasome inhibition results in a therapeutically beneficial antitumor, antiinflammatory, antiviral, or antiparasitic effect.

Predicting Response to Therapy

In particular embodiments, the level of proteasomal peptidase activity in a leukemia patient can be used to predict response to therapy for leukemia patients of a particular risk category according to cytogenetic analysis (e.g., ISCN standards). For example, AML patients having good cytogenetics or in the good-risk category are treated with chemotherapy, while patients having bad or very bad cytogenetics or are in the highest risk categories are treated with bone marrow transplants. However, approximately half of AML patients have “normal” cytogenetics and therefore fall into an intermediate risk group, wherein the treatment and the response thereto can vary considerably (Marcucci et al., Curr Opin Hematol 12(1):68-75, 2005). As shown herein, a correlation exists between the Cas-L activity and the response to treatment for AML patients having intermediate-risk cytogenetics. In particular, a Cas-L activity of greater than 3 pmol AMC/sec/mL plasma is associated with a poor response to chemotherapy. Further, that level of Cas-L activity could be used in determining that a bone marrow transplant is indicated.

Determining Prognosis

Provided herein are methods of using proteasomal peptidase activity levels in a test sample from a patient in conjunction with clinical factors other than proteasomal peptidase activity in determining the prognosis for a patient having a hematological disorder. In some embodiments, prognosis may be a prediction of the likelihood that a patient will survive for a particular period of time, or the prognosis is a prediction of how long a patient may live, or the prognosis is the likelihood that a patent will recover from a disease or disorder. There are many ways that prognosis can be expressed. For example prognosis can be expressed in terms of complete remission rates (CR), overall survival (OS) which is the amount of time from entry to death, disease-free survival (DFS) which is the amount of time from CR to relapse or death.

In certain embodiments high levels of certain proteasome peptidase activities are used as indicators of an unfavorable prognosis. According to the method, the determination of prognosis can be performed by comparing the measured proteasome peptidase activity levels to levels determined in comparable samples from healthy individuals or to levels known to corresponding with favorable or unfavorable outcomes. The absolute proteasome peptidase activity levels obtained may depend on an number of factors, including but not limited to the laboratory performing the assays, the assay methods used, the type of body fluid sample used and the type of disease a patient is afflicted with. According to the method, values can be collected from a series of patients with a particular disorder to determine appropriate reference ranges of each of the proteasome peptidase activities (i.e. Ch-L activity, Tr-L activity, Cas-L activity) for that disorder. One of ordinary skill in the art is capable of performing a retrospective study that compares the determined proteasome activity levels to the observed outcome of the patients and establishing ranges of levels for each activity that can be used to designate the prognosis of the patients with a particular disorder. For example, proteasome activity levels in the lowest range would be indicative of a more favorable prognosis, while proteasome activity levels in the highest ranges would be indicative of an unfavorable prognosis. Thus, in this aspect the term “elevated levels” refers to levels of proteasome activity that are above the range of the reference value. In some embodiments patients with “high” or “elevated” proteasome activity levels have activity levels that are higher than the median activity in a population of patients with that disease. In certain embodiments, “high” or “elevated” proteasome activity levels for a patient with a particular disease refers to levels that are above the median values for patients with that disorder and are in the upper 40% of patients with the disorder, or to levels that are in the upper 20% of patients with the disorder, or to levels that are in the upper 10% of patients with the disorder, or to levels that are in the upper 5% of patients with the disorder.

Because the proteasome peptidase activity levels in a test sample from a patient relate to the prognosis of a patient in a continuous fashion, the determination of prognosis can be performed using statistical analyses to relate the determined activity levels to the prognosis of the patient. A skilled artisan is capable of designing appropriate statistical methods. For example the methods of the present invention may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses.

In certain embodiments, the prognosis of AML, CLL, ALL, or MDS patients can be correlated to the clinical outcome of the disease using the proteasome peptidase activity levels and other clinical factors. Simple algorithms have been described and are readily adapted to this end. The approach by Giles et. al., British Journal of Hemotology, 121:578-585, hereby incorporated as reference, is exemplary. As in Giles et al., associations between categorical variables (e.g., proteasome activity levels and clinical characteristics) can be assessed via crosstabulation and Fisher's exact test. Unadjusted survival probabilities can be estimated using the method of Kaplan and Meier. The Cox proportional hazards regression model also can be used to assess the ability of patient characteristics (such as proteasome activity levels) to predict survival, with ‘goodness of fit’ assessed by the Grambsch-Therneau test, Schoenfeld residual plots, martingale residual plots and likelihood ratio statistics (see Grambsch, 1995; Grambsch et al, 1995). In some embodiments this approach can be adapted as a simple computer program that can be used with personal computers or personal digital assistants (PDA). The prediction of patients' survival time in based on their proteasome activity levels can be performed via the use of a visual basic for applications (VBA) computer program developed within Microsoft® excel. The core construction and analysis may be based on the Cox proportional hazard models. The VBA application can be developed by obtaining a base hazard rate and parameter estimates. These statistical analyses can be performed using a statistical program such as the SAS® proportional hazards regression, PHREG, procedure. Estimates can then be used to obtain probabilities of surviving from one to 24 months given the patient's covariates. The program can make use of estimated probabilities to create a graphical representation of a given patient's predicted survival curve. In certain embodiments, the program also provides 6-month, 1-year and 18-month survival probabilities. A graphical interface can be used to input patient characteristics in a user-friendly manner.

In some embodiments of the invention, multiple prognostic factors, including proteasome levels, are considered when determining the prognosis of a patient. For example, the prognosis of an AML, CLL, ALL, or MDS patient may be determined based on proteasome activity levels and one or more prognostic factors selected from the group consisting of cytogenetics, performance status, AHD (antecedent hematological disease), age, and diagnosis (e.g., MDS v. AML). In certain embodiments, other prognostic factors may be combined with the proteasome activity levels in the algorithm to determine prognosis with greater accuracy.

### EXAMPLE 1

### Variations in Proteasome Enzymatic Activities in Plasma of Patients with Chronic Lymphocytic Leukemia (CLL) and their Use in Predicting Clinical Behavior

Ch-L, Tr-L, and Cas-L activities in peripheral blood plasma from patients having chronic lymphocytic leukemia (CLL) were determined and correlated to clinical behavior. A fluorogenic kinetic assays using peptide-AMC (7-amino 4-methylcoumarin) substrates was used to measure the Ch-L, Tr-L, and Cas-L activities in the plasma of 226 patients with chronic lymphocytic leukemia (CLL) and 42 healthy individuals.

Ch-L, Tr-L, and Cas-L activities in peripheral blood plasma from patients were measured as follows. Frozen patient plasma samples were processed by thawing at room temperature and mixed thoroughly. 45 μL of thawed plasma was pipetted into a microfuge tube (Tube 1), and 22.5 μL plasma was pipetted into another microfuge tube (Tube 2). 5 μL 10% SDS was pipetted into Tube 1, and 2.5 μL 10% tween 20 was pipetted into Tube 2. Each tube was mixed and incubated at room temperature for 15 minutes resulting in processed samples. All buffers, substrate stock solutions, controls, and microwell plates were equilibrated to room temperature. The proteasome control was placed on ice immediately after thawing. Substrate working solutions (1 mM) were made by adding 950 μL buffer A (1×HEPES/0.05% SDS) to 50 μL stock solution of Ch-L and Cas-L substrates (20 mM Suc-LLVY-AMC and 20 mM Z-LLE-AMC) and mixed. 950 μL buffer B (1×HEPES/0.05% Tween 20) was added to 50 μL stock solution of Tr-L substrate (20 mM BZ-VGR-AMC) and mixed. Working solutions were protected from light. A microwell plate (containing 12 columns of wells) was set-up according to a plate map as follows: 30 μL buffer A was pipetted into the wells of columns 1, 4, 7, and 10 (to measure Ch-L activity) and 3, 6, 9, and 12 (to measure Cas-L activity). 30 μL buffer B was pipetted into the wells of columns 2, 5, 8, and 11 (to measure Tr-L). 10 μL buffer A or B served as blank controls. 10 μL of processed samples were pipetted into duplicate wells for each substrate (i.e., 6 wells per processed plasma sample), to the designated wells according to the plate map. 10 μL of substrate was added to the designated wells according to the plate map. The final concentration of substrate was 200 μM. Cleavage of substrate was detected using the SPECTRAmax GEMINI EM instrument with SoftMax Pro data Collection software. The instrument incubation chamber temperature was set to 37° C. Fluorescence excitation and emission wavelengths were 380 nm and 460 nm, respectively. Samples were read at 1 minute intervals over 30 minutes. Green fluorescence was detected in columns 1, 4, 7, and 10, representing Ch-L activity; blue fluorescence was detected in columns 3, 6, 9, and 12, representing Cas-L activity; and yellow fluorescence was detected in columns 2, 5, 8, and 11, representing Tr-L activity.

The enzymatic activities were expressed as pmol AMC/sec/mL plasma. Ch-L, Tr-L, and Cas-L activities were significantly (p<0.001) higher in the plasma of patients with CLL (medians: 1.47, 2.44, and 1.38 pmol AMC/sec/mL, respectively) than in healthy volunteers (medians: 0.80, 0.74, and 0.81 pmol AMC/sec/mL, respectively). Although Ch-L and Cas-L activities did not differ significantly between men and women with CLL, Tr-L activity was significantly higher in women (P=0.01). Rai stage correlated with Ch-L (P<0.001) but not Cas-L or Tr-L activity. Only Ch-L activity correlated with WBC count (P<0.001). μ2-microglobulin levels correlated strongly with Ch-L activity (R=0.40, P<0.001) and weakly with Cas-L activity (R=0.25, P 0.001) but not with Tr-L activity. Ch-L and Cas-L activities were both strong and independent predictors of survival when examined as continuous variables (P=0.02 for both), as well as when the median was used as a cut-off point (P=0.02 and P=0.03, respectively). Both Ch-L and Cas-L activities were independent of μ2-microglobulin in predicting survival, but both correlated with each other and were not independent of each other in predicting survival. There was no correlation between Tr-L activity and survival. These data suggest not only that proteasome activity as measured in the plasma of patients with CLL has important prognostic value, but also that CLL patients may benefit from proteasome inhibition therapy that specifically targets Ch-L or Cas-L activities.

### EXAMPLE 2

### Variations in Proteasome Enzymatic Activities in Plasma of Patients with Acute Myeloid Leukemia and Myelodysplastic Syndrome and their Use in Predicting Clinical Behavior

Ch-L, Tr-L, and Cas-L activities in peripheral blood plasma from patients having acute myeloid leukemia (AML) and myelodysplastic syndrome (MDS) were determined and correlated to clinical behavior. A fluorogenic kinetic assays using peptide-AMC (7-amino 4-methylcoumarin) substrates was used to measure the Ch-L, Tr-L, and Cas-L activities in the plasma of 188 patients with acute myeloid leukemia (AML), 58 patients with myelodysplastic syndrome (MDS), and 42 healthy individuals.

Ch-L, Tr-L, and Cas-L activities in peripheral blood plasma from patients were measured as in Example 1. Significantly higher (P<0.001) Ch-L, Tr-L, and Cas-L activities were seen in AML patients (medians: 1.39, 1.51, and 2.40 pmol AMC/sec/mL, respectively) and MDS patients (medians: 1.16, 1.40, and 1.67 pmol AMC/sec/mL, respectively) than in healthy volunteers (medians: 0.80, 0.74, and 0.81 pmol AMC/sec/mL, respectively). The difference in Cas-L activity between AML and MDS patients was significant (P<0.001). While there was no significant difference between Ch-L and Cas-L activities in healthy controls, there was a significant difference between the 2 activities in both AML and MDS. Cas-L and Ch-L, but not Tr-L, correlated with WBC count and lactic dehydrogenase in AML and MDS patients. In AML patients, higher levels of Ch-L and Cas-L were associated with poor response to a variety of therapies (P=0.004 and P=0.001, respectively). Cas-L correlated strongly with survival in AML patients when used as an activity-dependent variable (P<0.001) or when the median was used as a cut-off (P=0.004). This was independent of cytogenetic abnormalities, age, and performance status. Patients with intermediate-risk cytogenetic abnormalities and Cas-L activity >3 pmol AMC/sec/mL had significantly shorter survival (P=0.04). Ch-L activity was also predictive of survival in AML independent of age and cytogenetic and performance status, but not independent of Cas-L. In MDS, higher levels of Cas-L, but not Ch-L, correlated with shorter survival and this was independent of cytogenetic abnormalities. The increased cell-free circulating proteasome activities most likely reflect the leukemic cells and may be a marker not only for disease, but also potentially for monitoring therapy. These data also suggest that patients with AML may benefit differentially from proteasome inhibitors depending on the specific therapeutic effect of the inhibitor.

### EXAMPLE 3

### Measurement and Clinical Relevance of Proteasome Enzymatic Activity in Plasma of Patients with Acute Lymphoblastic Leukemia (ALL)

Ch-L, Tr-L, and Cas-L activities in peripheral blood plasma from patients having acute lymphoblastic leukemia (ALL) were determined and correlated to clinical behavior. A fluorogenic kinetic assays using peptide-AMC (7-amino 4-methylcoumarin) substrates was used to measure the Ch-L, Tr-L, and Cas-L activities in the plasma of 57 patients having acute lymphoblastic leukemia (ALL) and 42 healthy individuals.

Ch-L, Tr-L, and Cas-L activities in peripheral blood plasma from patients were measured as in Example 1. Significantly higher (P<0.001) Ch-L, Tr-L, and Cas-L activities were detected in patients with ALL (medians: 1.40, 2.06, and 2.04 pmol AMC/sec/mL, respectively) than in healthy volunteers (n=42) (medians: 0.80, 0.74, and 0.81 pmol AMC/sec/mL, respectively). While there was no significant difference between Ch-L and Cas-L activities in healthy controls, there was a significant difference between the 2 activities in patients with ALL. Cas-L, Tr-L, and Ch-L all correlated positively with lactic dehydrogenase (P<0.01). However, ALL patients with Cas-L activity ≧2.7 pmol AMC/sec/mL had significantly longer survival (P=0.01) than did patients with lower activity. The increased cell-free circulating proteasome activities most likely reflect the leukemic cells and may represent a marker not only for the disease, but also for monitoring therapy. These data also suggest that patients with ALL may benefit differentially from proteasome inhibitors depending on the specific therapeutic properties of the inhibitor.

### EXAMPLE 4

### Effect of Proteasome Inhibitor on Peptidase Activity

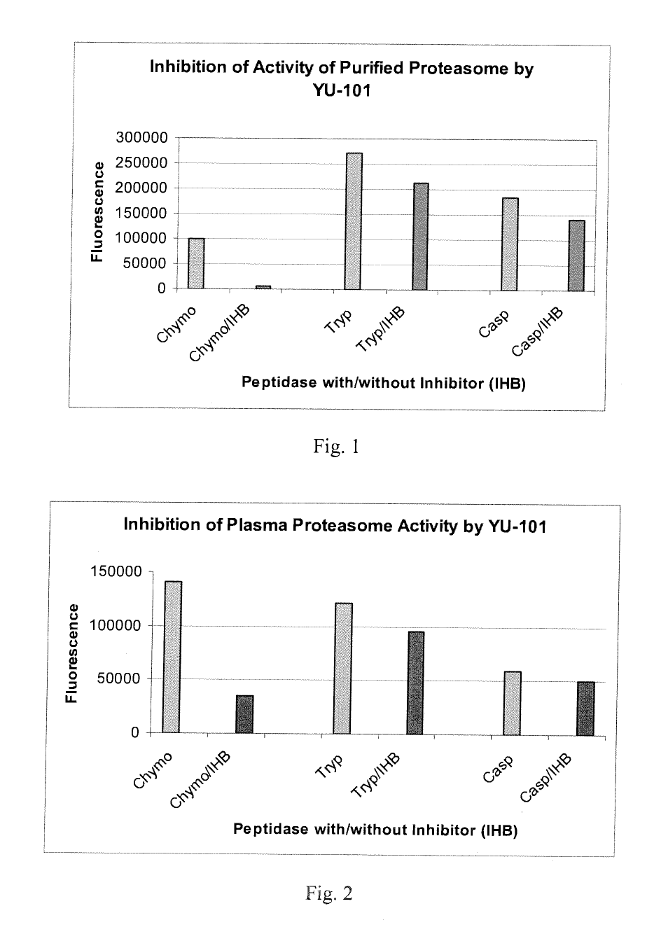

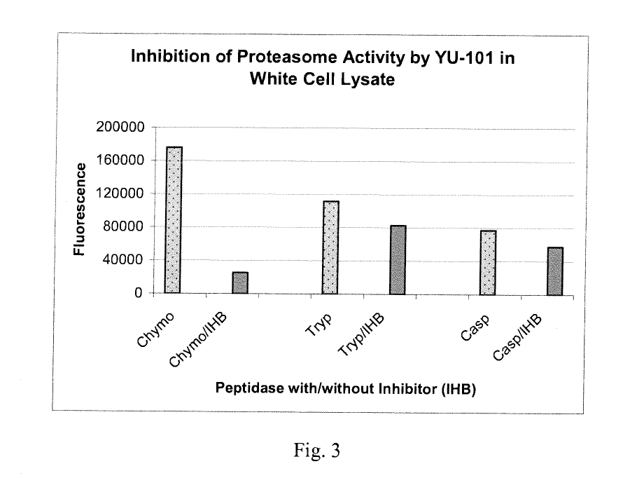

Ch-L, Tr-L, and Cas-L peptidase activities were determined in the presence and absence of proteasome inhibitor in samples of purified proteasome, plasma, and white blood cell lysate, the latter two samples were from normal individuals. Fluorogenic assays using peptide-AMC (7-amino 4-methylcoumarin) substrates was used to measure the Ch-L, Tr-L, and Cas-L activities at a concentration of 200 μM. YU-101 was used as the inhibitor at a final concentration of 0.4 μg/mL. 70 μL of sample (purified proteasome, plasma, or white blood cell lysate) was combined with 1 μL inhibitor solution and incubated at room temperature for 15 minutes. Ch-L, Tr-L, and Cas-L activities were determined as in Example 1. Peptidase activity levels in the presence and absence of inhibitor in each sample type are shown in FIGS. 1, 2, and 3 for purified proteosome, plasma, and white blood cell lysate, respectively. All three sample types exhibited a similar inhibition profile, in which YU101 is a strong inhibitor for chymotrypsin-L activity and a weaker inhibitor for trypsin-L and caspase-L activity.

A dose response study was conducted to determine the effect of concentration of YU-101 on Ch-L activity as assayed in plasma from an AML patient. YU-101 (200 ng/μL) was serially diluted 1:4 in HEPES buffer to produce solutions having concentrations of 0.78, 3.125, 12.5, 50, and 200 ng/μL for dose response experiments. 70 μL of plasma from an AML patient was combined with 1 μL inhibitor solution and incubated at room temperature for 15 minutes. Ch-L activity was determined as in Example 1. The data from a representative dose response experiment are shown below. These data show that YU-101 inhibition of Ch-L activity in plasma of an AML patient is dose-dependent.

TABLE 1
YU-101 Inhibition of proteasome activity in plasma from an
AML patient.

| Approx. YU-101 | | |
|---|---|---|
| concentration (ng/μL) | Activity | % Inhibition |
| 0.00156 | 233781 | 0.00% |
| 0.00625 | 212786 | 8.98% |
| 0.025 | 143991 | 38.41% |
| 0.1 | 87573 | 62.54% |
| 0.4 | 71162 | 69.56% |

A dose response study was conducted to determine the effect of concentration of YU-101 on Ch-L activity in a whole blood lysate from a normal individual. YU-101 (200 ng/μL) was serially diluted 1:2 in HEPES buffer to produce solutions having concentrations of 6.25, 12.5, 25, 50, and 100 ng/μL for dose response experiments. 70 μL of plasma from an AML patient was combined with 1 μL inhibitor solution and incubated at room temperature for 15 minutes. Ch-L activity was determined as in Example 1. The data from a representative dose response experiment are shown below. These data show that YU-101 inhibition of Ch-L activity in a sample of whole blood cell lysate from a normal individual is dose-dependent.

TABLE 2
YU-101 Inhibition of proteasome activity in whole blood from a
normal individual.

| Approx. YU-101 | | |
|---|---|---|
| concentration (ng/μL) | Activity | % Inhibition |
| 0.0 | 95006 | 0.00% |
| 0.0125 | 92002 | 3.2% |
| 0.025 | 72252 | 24.0% |
| 0.05 | 58544 | 38.4% |
| 0.1 | 17070 | 82.0% |
| 0.2 | 7024 | 92.6 |

### EXAMPLE 5

### Specificity of Proteasomal Enzymatic Assay

The specificity of the proteasomal enzymatic assay was determined by comparing human pancreatic chymotrypsin activity to proteasomal chymotrypsin-like (Ch-L) activity in the assay described in Example 1. Briefly, chymotrypsin or chymotrypsin-like activity was determined using a fluorogenic assay, which detects the fluorescence generated by cleavage of the substrate Suc-LLVY-AMC (200 μM) in a sample of isolated human pancreatic α-chymotrypsin (Sigma, catalog #C-8946) in the presence and absence of proteasome inhibitor (YU-101, 0.4 μg/mL) or pancreatic chymotrypsin inhibitor a 1-anti-chymotrypsin (Sigma, catalog #A9285). Chymotrypsin-like activity in a sample of purified proteasomes was assayed in parallel.

Pancreatic α-chymotrypsin (219 μg/vial) was reconstituted in 200 μL HEPES buffer to a final concentration of 1.1 μg/μL. Pure human 20S proteasome (Biomol International, Cat# PW8720-0050) was diluted 1:100 with HEPES buffer to a final concentration of 10 μg/μL. The α1-anti-chymotrypsin (1 mg/vial) was reconstituted in 500 μL DI water to a final concentration of 2 μg/μL. A working solution of YU-101 was made by reconstituting YU-101 to a final concentration of 0.2 μg/μL.

Samples containing diluted 20S proteasome or reconstituted α-chymotrypsin with or without inhibitor (either α1-anti-chymotrypsin or YU-101) were incubated at room temperature for 15 minutes. For samples with inhibitor, 1 μL of anti-chymotrypsin (2 μg/μL) or YU-101 (0.2 μg/μL) was added to 10 μL α-chymotrypsin or 20S proteasome. 10 μL of each sample was then combined with 30 μL assay buffer and 10 μL suc-LLVY-AMC substrate for a total reaction volume of 50 μL (resulting in a final concentration of α1-anti-chymotrypsin of 40 ng/μL and a final concentration of YU-101 of 4 ng/μL). The release of AMC was monitored for 30 minutes. The determined Ch-L and chymotrypsin activities are shown in Table 3 below.

TABLE 3
Chymotrypsin-like activity of purified proteasomes and
pancreatic chymotrypsin.

| Sample | | Vₘₐₓ Ch-L |
|---|---|---|
| No. | Sample | (fluorescence) |
| 1 | Pancreatic α-chymotrypsin | No activity |
| 2 | Pancreatic α-chymotrypsin + α1-anti- | No activity |
| | chymotrypsin (chymotrypsin inhibitor) | |
| 3 | Pancreatic α-chymotrypsin + YU101 | No activity |
| | (proteasome inhibitor) | |
| 4 | Purified 20S proteasome | 112,516 |
| 5 | Purified 20s proteasome + α1-anti- | 121,813 |
| | chymotrypsin (chymotrypsin inhibitor) | |
| 6 | Purified 20S proteasome + YU101 | No activity |
| | (proteasome inhibitor) | |

These data suggest that the proteasome assay is specific for proteasomal Ch-L activity over pancreatic chymotrypsin activity and support that the Ch-L activity in plasma results from the proteasomal Ch-L released from cell proteasomes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

That which is claimed is:

1. A method of diagnosing a disease or disorder other than a pancreatic disease or disorder, cystic fibrosis, or renal failure in a subject, said method comprising,
determining the level of one or more proteasomal peptidase activities in a test sample from said subject, said peptidase activities selected from the group consisting of chymotrypsin-like activity (Ch-L), trypsin-like activity (Tr-L), and caspase-like activity (Cas-L), and
using the determined levels of one or more proteasomal peptidase activities in conjunction with clinical factors other than proteasomal peptidase activity to diagnose the presence of said disease or disorder in said patient.

2. A method according to claim 1, wherein said test sample is an acellular body fluid sample.

3. A method according to claim 1, wherein said test sample is a cell-containing sample.

4. A method according to claim 1, wherein an increase in the level of said one or more proteosomal peptidase activities relative to the level of the corresponding proteosomal peptidase activity in a comparable sample from one or more healthy individuals is used with clinical factors other than proteasomal peptidase activity to diagnose the presence of said disease or disorder in said patient.

5. A method according to claim 1, wherein said disease or disorder is selected from the group consisting of a cancer, a proliferative hematological disorder, an inflammatory disease, an autoimmune disease, or a degenerative disease.

6. A method according to claim 5, wherein said disease or disorder is a proliferative hematological disorder.

7. A method according to claim 6 wherein an increase in the level of said one or more proteosomal peptidase activities relative to the level of the corresponding proteosomal peptidase activity in a comparable sample from one or more healthy individuals is a factor favoring diagnosis of a proliferative hematological disorder while the same or a decrease in the level of said one or more proteosomal peptidase activities relative to the level of the corresponding proteosomal peptidase activity in a comparable sample from one or more healthy individuals is a factor against diagnosis of a proliferative hematological disorder.

8. A method according to claim 6, wherein the proliferative hematological disorder is selected from the group consisting of chronic lymphocytic leukemia (CLL), acute myeloid leukemia (AML) myelodysplastic syndrome (MDS), and acute lymphoblastic leukemia (ALL).

9. A method according to claim 6, wherein said proliferative hematological disorder is acute myeloid leukemia (AML).

10. A method according to claim 6, wherein said proliferative hematological disorder is myelodysplastic syndrome (MDS).

11. A method according to claim 6, wherein said proliferative hematological disorder is acute lymphoblastic leukemia (ALL).

12. The method according to claim 2, wherein said acellular body fluid is selected from the group consisting of serum and plasma.

13. A method for predicting the response to therapy of leukemia patient having intermediate-risk cytogenetic abnormalities, said method comprising
determining the level of proteasomal caspase-like activity (Cas-L) in a test sample from said patient,
using said level to predict the response of said patient to therapy.

14. A method according to claim 13, wherein said test sample is a cell-containing sample.

15. A method according to claim 13, wherein said test sample is an acellular body fluid sample.

16. A method according to claim 15 wherein said level of Cas-L activity is compared to a cutoff value determined from the level of Cas-L activity present in a comparable sample from healthy individuals, and wherein an increase or decrease in the patient value relative to the cutoff value is used to predict the response of said patient to therapy.

17. A method according to claim 16, wherein said cutoff value is 3 pmol AMC/sec/mL and a level of Cas-L activity in said acellular test sample greater than the cutoff value is associated with a poor response to chemotherapy.

18. A method according to claim 13, wherein said leukemia patient has AML.

19. A method of monitoring proteasome inhibitor drug therapy in a patient, said method comprising,
obtaining a sample of an acellular body fluid from a patient previously administered a proteasome inhibitor;
determining the level of one or more proteasomal peptidase activities in a sample of an acellular body fluid from said patient, said peptidase activities selected from the group consisting of chymotrypsin-like activity (Ch-L), trypsin-like activity (Tr-L), and caspase-like activity (Cas-L), and
comparing the level of activity in said sample to a control level of activity for each peptidase determined in a comparable sample of acellular body fluid obtained from said patient prior to the administration of said proteasome inhibitor or obtained from a one or more healthy individuals.

20. A method according to claim 19, wherein said control level of activity for each peptidase is determined in a comparable sample of acellular body fluid obtained from said patient prior to the administration of said proteasome inhibitor.

21. A method according to claim 19, wherein the level of one or more proteasomal peptidase activities is determined for acellular body fluid samples taken from the patient at two or more time points following administration of the proteosome inhibitor.

22. The method according to claim 19, wherein said body fluid is selected from the group consisting of serum and plasma.

23. A method of determining a prognosis of a patient having a disease or disorder, wherein said method comprises,
determining the level of one or more proteasomal peptidase activities in a test sample from said subject, said peptidase activities selected from the group consisting of chymotrypsin-like activity (Ch-L), trypsin-like activity (Tr-L), and caspase-like activity (Cas-L), and
using the levels of one or more proteasomal peptidase activities determined for the patient in conjunction with clinical factors other than proteasomal peptidase activity to determine a prognosis for the patient, wherein
a determined level of said one or more proteosomal peptidase activity in said patient sample that is higher than a reference value reflects a negative prognosis for the patient, and
a determined level of said one or more proteosomal peptidase activity in said patient sample that is the same or substantially the same as a reference value reflects a positive prognosis for the patient.

24. A method according to claim 23, wherein the disease or disorder is other than a pancreatic disease or disorder, cystic fibrosis, or renal failure.

25. The method according to claim 23, wherein the reference value is the corresponding proteosomal peptidase activity in a comparable sample from one or more healthy individuals.

26. A method according to claim 23, wherein said test sample is a cell-containing sample.

27. A method according to claim 23, wherein said test sample is an acellular body fluid sample.

28. The method according to claim 27, wherein said body fluid is selected from the group consisting of serum and plasma.

29. The method of claim 23, wherein prognosis is selected from the group consisting of survival rate, 5-year survival rate, and complete remission duration (CRD).

30. The method according to claim 23, wherein said disease or disorder is a proliferative hematological disorder.

31. The method according to claim 30, wherein said proliferative hematological disorder is selected from the group consisting of AML, CLL, ALL and MDS and said prognosis is survival rate.

32. A method of determining diagnosis or prognosis of a disease or disorder other than a pancreatic disease or disorder, cystic fibrosis, or renal failure in a subject, said method comprising,
determining in a test sample from said subject a peptidase activity level for one or more substrates, wherein each of said substrates is cleaved by a peptidase activity selected from the group consisting of proteasomal chymotrypsin-like activity, (Ch-L), proteasomal trypsin-like activity (Tr-L), and proteasomal caspase-like activity (Cas-L), and
using the determined peptidase activity level for one or more substrates in conjunction with clinical factors other than proteasomal peptidase activity to determine the diagnosis the presence of said disease or disorder in said patient or determine the prognosis of said patient.

33. A method according to claim 32, wherein said test sample is an acellular body fluid sample.

34. A method according to claim 32, wherein said test sample is a cell-containing sample.

35. A method according to claim 32, wherein said disease or disorder is a proliferative hematological disorder.

36. A method according to claim 35 wherein an increase in the level of said one or more proteosomal peptidase activity relative to the level of the corresponding proteosomal peptidase activity in a comparable sample from one or more healthy individuals is a factor favoring diagnosis of a proliferative hematological disorder while the same or a decrease in the level of said one or more proteosomal peptidase activities relative to the level of the corresponding proteosomal peptidase activity in a comparable sample from one or more healthy individuals is a factor against diagnosis of a proliferative hematological disorder.

37. A method according to claim 35, wherein said proliferative hematological disorder is selected from the group consisting of chronic lymphocytic leukemia (CLL), acute myeloid leukemia (AML) myelodysplastic syndrome (MDS), and acute lymphoblastic leukemia.
